(19) United States
(12) Patent Application Publication
(10) Pub. No.: US 2005/0288638 A1
(43) Pub. Date: Dec. 29, 2005

(54) IV tubing with valve and method for use

(76) Inventor: Thomas Michel III, Dacula, GA (US)

Correspondence Address: LITMAN LAW OFFICES, LTD, PO BOX 15035, CRYSTAL CITY STATION, ARLINGTON, VA, 22215, US

(21) Appl. No.: 10/873,136

(22) Filed: Jun. 23, 2004

(51) Int. Cl. A61M 5/00

(52) U.S. Cl. 604/252; 604/80

(57) ABSTRACT

An intravenous tubing and air-separating valve arrangement that prevents the entry of air into the vascular system of a patient during intravenous procedures. Applicable for both pumped and non-pumped intravenous procedures, the effectiveness of the arrangement is dependent on the position of the air-separating valve relative to the patient or pump.

### BACKGROUND OF THE INVENTION

1. Field of the Invention

The present invention generally relates to medical equipment. More specifically, the present invention is drawn to an I.V. tube and valve arrangement that minimizes the chance of bubbles entering a patient's bloodstream during an intravenous dosing or feeding procedure.

2. Description of the Related Art

In hospitals and medical clinics throughout the world, thousands, if not millions of patients, daily receive medicine or other life-saving fluids intravenously (or intra-arterially). Basically, intravenous apparatus include a bag containing the fluid to be fed to the patient, tubing for transferring the fluid from the bag and an I.V. catheter for insertion in the patient (usually the arm). During the intravenous procedure, medical personnel must be constantly vigilant to prevent air bubbles which may be trapped in the fluid from entering the patient's vascular system. Such air bubbles can cause an air embolism, which air embolism can lead to serious complications, even death. Systems have been developed to separate the air bubbles from the intravenously-fed fluid and to signal medical personnel when air bubbles are about to enter the patient's system. The above-mentioned systems are usually relatively costly, complicated and require an inordinate amount of time in that personnel must respond to the signal to rid the system of air bubbles. The art would certainly welcome an uncomplicated, relatively inexpensive and efficient arrangement for ridding I.V. systems of worrisome air bubbles.

There are many devices in the related art drawn to I.V. apparatus and systems. For example, U.S. Patent Application Publication 2003/0055375 A1 (Holst et al.) discloses a method for compensating for pressure differences across valves in a cassette-type I.V. pump. The Holst et al. arrangement is complicated and no mention is made of an air-separating valve position relative to the patient or the pump.

U.S. Pat. No. 4,784,644 (Sawyer et al.) is drawn to a valve catheter and method for the prevention of air into the body of a patient. The patentees do not contemplate the position of an air-separating valve relative to the patient or the pump.

U.S. Pat. No. 5,935,105 (Manning et al.) shows a system that eliminates air from an intravenous line. The system requires a digital controller and an array of valves. No mention is made of the valves proximity to the patient or to a pump.

U.S. Pat. No. 5,308,333 (Skakoon) discloses an intravenous pump that incorporates an air-eliminating filter. The patentee does not contemplate utilizing the filter without the pump.

U.S. Pat. No. 6,193,689 B1 (Woodward) is drawn to an intravenous flow regulator that employs an air separator valve. The patentee does not contemplate the advantages attained in positioning the valve close to the patient or close to the pump.

None of the above inventions and patents, taken either singly or in combination, is seen to disclose and intravenous tube and valve arrangement as will subsequently be described and claimed in the instant invention.

### SUMMARY OF THE INVENTION

The present invention is an I.V. tubing and air-separating valve (or bubble separator) arrangement that prevents the entry of air into the vascular system of a patient during intravenous procedures. Applicable for both pumped and non-pumped intravenous procedures, the effectiveness of the arrangement is dependent on the position of the air-separating valve relative to the patient or pump.

If a pump is utilized, the valve is positioned in the feed line immediately adjacent and upstream of the pump. This arrangement would insure that all air is separated from the fluids before the fluids enter the pump housing. Thus, no air is available in the pump to stop the pump and sound an alarm. Consequently, only the intended fluids are pumped into the patient's vascular system.

If the I.V. system is used without a pump, the air-separating valve is disposed close to the patient at a position immediately adjacent to and upstream of the I.V. catheter. As in the arrangement discussed above, no air can reenter the line and only the intended fluids are available to enter the patient via the catheter.

Accordingly, the instant invention presents an intravenous feed arrangement that is effective to ensure the safety of the patient as related to air embolisms. The invention is also effective to save time and money in that medical personnel will not have to respond to frequent air-bubble alarms nor will costly fluids, intended to be given to the patients, be wasted in the process of purging the air. The patient benefits both physically and psychologically since the fluids are efficiently fed through the system and no dreaded air-bubbles are visibly seen as the fluids enter the patient.

The invention provides improved elements and arrangements thereof for the purposes described which are inexpensive, dependable and fully effective in accomplishing their intended purposes.

A clear understanding of the present invention will become readily apparent upon further review of the following specification and drawings. dr

### BRIEF DESCRIPTION OF THE DRAWINGS

FIG. 1 is an environmental, perspective view of an I.V. tubing an air-separating valve in a gravity-fed arrangement according to the present invention.

FIG. 2 is an environmental, perspective view of an I.V. tubing an air-separating valve in a pump-fed arrangement according to the present invention.

FIG. 3 is a cross-sectional view of a bubble separator (Prior Art).

Similar reference characters denote corresponding features consistently throughout the attached drawings.

### DETAILED DESCRIPTION OF THE PREFERRED EMBODIMENTS

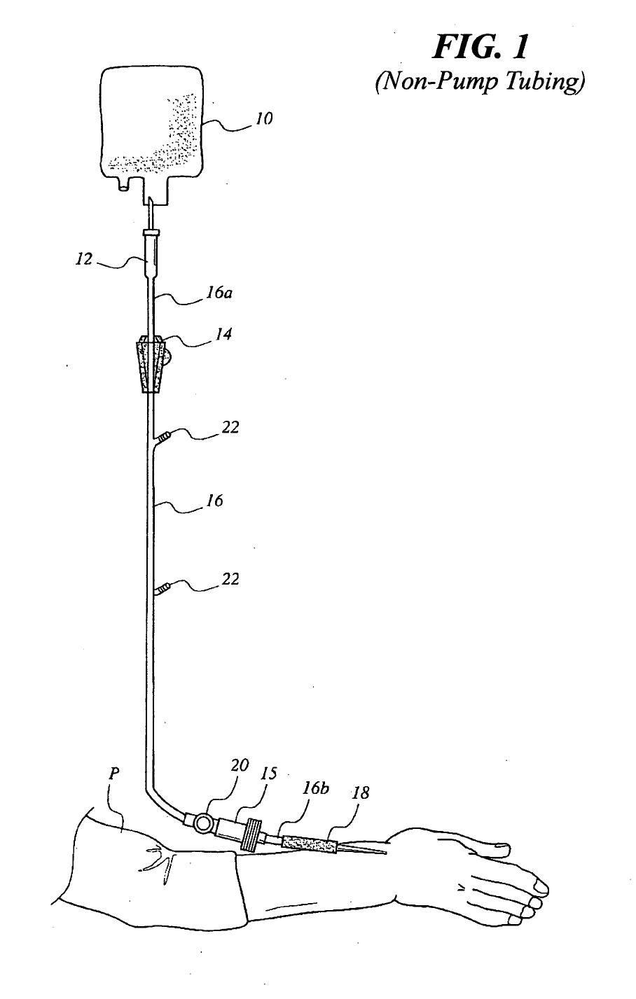

Attention is first directed to FIG. 1 wherein an I.V. system of the gravity fed type is illustrated. The system includes the conventional I.V. bag 10, drip chamber 12 and feed clamp 14. Tubing 16 has a distal end 16a opening into drip chamber 12. The proximate end 16b opens into a conventional catheter 18 via a conventional luer lock connector 15. A bubble separator 20 is positioned as close as possible to catheter 18. The only limitation as to closeness is the ability to secure the catheter to the patient P. Additional fluids may be supplied via auxiliary ports 22.

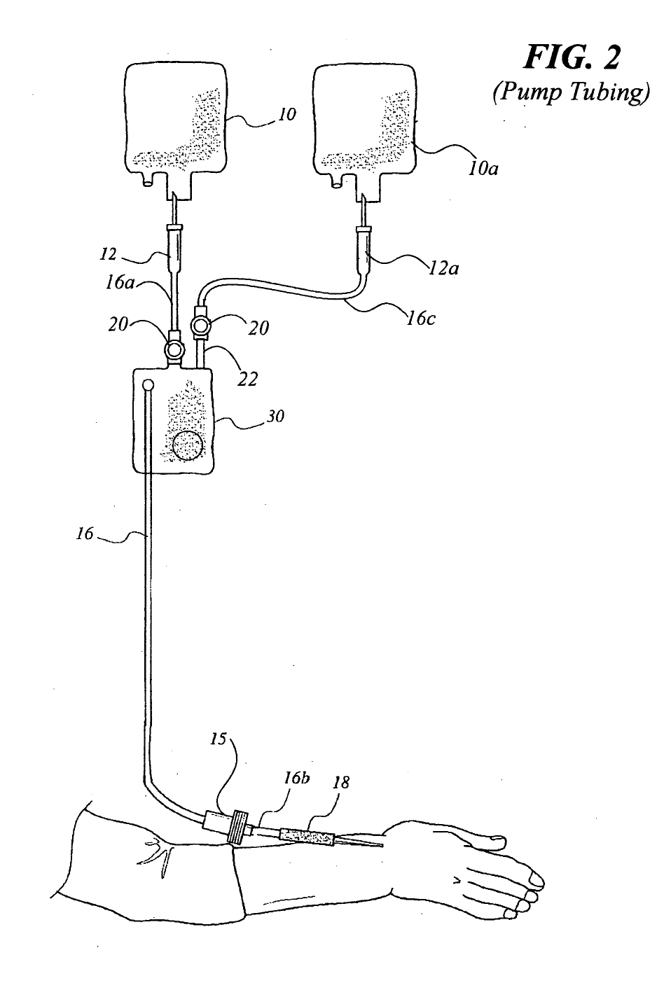

When a pump is utilized (FIG. 2), bubble separator 20 is positioned upstream of and immediately adjacent to the cassette housing 30. If fluids are to be provided via auxiliary port(s) 22, another bubble separator must be provided at the auxiliary port. Fluids are supplied to the auxiliary port from bag 10a via drip tube 12a and tubing 16c.

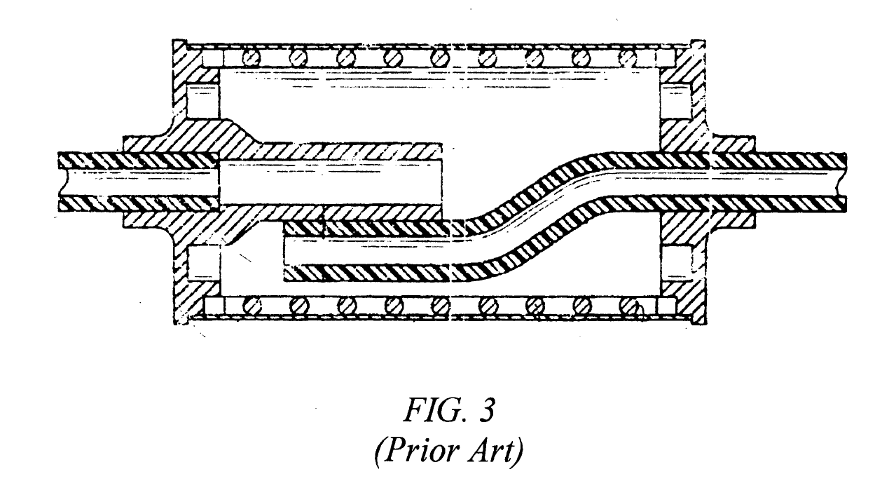

FIG. 3 is illustrative of one of the many prior art bubble separators that are available for use in Applicant's inventive arrangement. The operation of the bubble separator is completely explained in U.S. Pat. No. 6,193,689 B1 (Woodward), which is cited above.

It is to be understood that the present invention is not limited to the embodiment described above, but encompasses any and all embodiments within the scope of the following claims.

1. An intravenous feeding system for feeding fluids to a patient comprising:
a flexible bag for containing fluids to be fed to the patient;
a drip chamber attached to and in fluid communication with said flexible bag;
a feed tube, said feed tube having a distal end and a proximate end, said distal end attached to and in fluid communication with said drip chamber;
a catheter adapted to be inserted into the patient, said proximate end of said feed tube attached to and in fluid communication with said catheter; and
a bubble separator, said bubble separator positioned in said feed tube upstream of and immediately adjacent to said catheter.

2. An intravenous feeding system as recited in claim 1, further including a luer lock positioned between said bubble separator and said catheter, wherein said luer lock is in abutment with said catheter and said bubble separator is in abutment with said luer lock.

3. An intravenous feeding system for feeding fluids to a patient comprising:
a flexible bag for containing fluids to be fed to the patient;
a drip chamber attached to and in fluid communication with said flexible bag;
a feed tube, said feed tube having a distal end and a proximate end, said distal end attached to and in fluid communication with said drip chamber;
a catheter adapted to be inserted into the patient, said proximate end of said feed tube attached to and in fluid communication with said catheter;
a cassette housing attached to said feed tube;
a first fluid pump disposed in said cassette housing; and
a bubble separator, said bubble separator positioned in said feed tube upstream of and immediately adjacent to and in abutment with said cassette housing.

4. An intravenous feeding system as recited in claim 3, further including an auxiliary port disposed in said cassette housing, wherein a second bubble separator is positioned immediately upstream of an in abutment with said auxiliary port.

5. A method of feeding fluids intravenously to a patient comprising:
providing a bag containing fluids to be fed to the patient;
providing a feed tube for feeding fluids from said bag to the patient;
providing a bubble separator for separating air from said fluids; and
positioning said bubble separator in said feed tube upstream of and close to said patient to effectively and efficiently separate air bubbles from the fluid.

6. A method of feeding fluids intravenously as recited in claim 5, including the steps of;
providing a catheter in said feed tube for insertion into the patient;
providing a luer lock immediately upstream and in abutment with said catheter; and
positioning said bubble separator immediately upstream and in abutment with said luer lock.

7. A method of feeding fluids intravenously as recited in claim 5, including the steps of:
providing a catheter in said feed tube for insertion into the patient;
providing a luer lock immediately upstream and in abutment with said catheter;
providing a cassette housing in said feed tube upstream of said luer lock, said cassette housing having a fluid pump disposed therein;
positioning said bubble separator immediately upstream of and in abutment with said cassette housing.

8. A method of feeding fluids intravenously as recited in claim 6, including the steps of:
providing an auxiliary port in said cassette housing;
positioning a second bubble separator immediately upstream of an in abutment with said auxiliary port.
